SEQUENCE LISTING (8 sequences)

SEQ ID NO: 1 - DNA, 706 bp, Aequorea victoria
  CDS (8)..(706)
cgccaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataa

SEQ ID NO: 2 - protein, 232 aa, Aequorea victoria
MESDESGLPAMEIECRITGTLNGVEFELVGGGEGTPEQGRMTNKMKSTKGALTFSPYLLSHVMGYGFYHFGTYPSGYENPFLHAINNGGYTNTRIEKYEDGGVLHVSFSYRYEAGRVIGDFKVMGTGFPEDSVIFTDKIIRSNATVEHLHPMGDNDLDGSFTRTFSLRDGGYYSSVVDSHMHFKSAIHPSILQNGGPMFAFRRVEEDHSNTELGIVEYQHAFKTPDADAGEE

SEQ ID NO: 3 - DNA, 25 bp, Artificial Sequence
  backward primer
ccctcgagttattcttcaccggcat

SEQ ID NO: 4 - DNA, 46 bp, Artificial Sequence
  forward primer F1
tccctctatcacctgtgtgaggagggcctttatggagagcgacgag

SEQ ID NO: 5 - DNA, 46 bp, Artificial Sequence
  forward primer F2
gatggcagtggaggatgaagaagctgggtgctccctctatcacctg

SEQ ID NO: 6 - DNA, 46 bp, Artificial Sequence
  forward primer F3
ggatcccgccaccatgttcaaatgccgccgatggcagtggaggatg

SEQ ID NO: 7 - DNA, 8288 bp, Artificial Sequence
  pZ?GFP plasmid as a vector
cagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacggtcgcccagcaattcctgcagcccgggggatctgtccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtcgacggtatcgataagcttgataattctgcgcgcgcagattccgtgtgggcctggtaatatcttaccattttaagctattttaagattagggaaaaaaattaaataaaactactttaaaattactttccttaaaaagcagcatatttatttagtttttgtttaactggctcaagattgtaatattttacttttacatgtttttttaagtacctaaatgtgttcaaaagtaaagtatgttattgtttttttttttttaatgttagctaagctttaaattgtaatatattacttttaaaagtgactttttaaaaatgtaaataaaaaagtaactataaatgtgtaatgcatgttatttctgtgggtttttaattttcatctcaactgggcttacaattttaacataataaaaatcaaatcaaatcaaatcaggcccttatttgtcacatacactcttgtatactgaaataaactaaatgttgagaaaaatttaaatacaccaaaataacgcataccttttttgtaattacctaagtaaaacatttagttccttttttaagtgactcaagattgtaatctattaattgtaaaagataaaactagtgatcaatctgacgtgcttctatgcagcctgtgagattaattgtgatgtttaattattaaacaatttaaatggatcaaacatttcacatgtgcttattgtgtccaacaaaaccagcagaagcacttaaaaatattgcatgacaacatgagtttttaatgtaaattatatatacttcctaaacgaagttaatcatatttctaaaataataatgtcattgaaatatttaaataaatgataagctttagttgattcaacataaaattgaaagcaataaggactttttttttttacagcggtgaagcaaaagcatgtaataatcatataatataataaataaataaataaataatggctcttggtgcaaatatgaacactttaaataaatatagaataaatatgatcaataagtcataacgacgtgttttatgttgattataatttaaacatgtttctaacgcaagttttaaaaactgttttaaagtattattttaagttataagatcaatttgactcaacattttgtagtgaaaatatgttgtgtaaaattaatataatatacactataaaaatgcagggttccacacaatacattaatgttgtcccaacacaaatcgattaagttaacaaatatatgtggattgaacataaaaaattaagttgtcccaatgaaatctcaagaattgtgttgtttcagctcattttaaataagtagttttaacaagtaaaaaataacatatttttgagtgtgtatatatatatattacaaaatataataatataaaatgtaaaagatataaaccatgctacatatatttaagagctattttgcacattacttttttgcaagccattttagtgttttttttacaaatatatattagctaaatatacaatatcttaaatatacaattaatcagacatgcaattaatgcatttaacctacataaaaatacatgatcaacatatgttttatgctgattacaactaaatcaaacgtaatgaaattaatttataaaagcgatttaaatatgtcaatattttaagttaaaatcacttgaaacctactataaatgaacttaaattaaaaaaatctcctttaattccaatgcaacagtgttgttttttcagctgaggacccccggtgcagatggttggcccccggcccgctattctaaggaagcctttcttaattttagggcggcgctgcatgcgctcagatcagattcacgggtggagagggttattttctcttttttattttttcttgcctgctcatatttgggcctgtgtgaaccccaggctccgcctcggcccccaggagccgctcttatttaaaggggatcctcgccttcagtcttcgtcccagtggaagcatcagtcttgtgataacaacttggtgagtcctacagaccagcgctcgactctaggctttaaatgcattgatttctctggattagctaccgggagagttttaactttgcttattagtcttgtgctcgaactgctaggtttaaagagtgttttctctaatgcatgcatgaagcgacagctgaaaaccagaaatagagcgtttgtcctcctgttaaactctagcctttctttggcatttgtcagtccgagtcttcagctgccggcaggagattgggtttcagtaacacttgctgagcacaaactacacgtgctttcttcagatttaattcattattaatcattctagatttaaatgatgttcttactcattgctgattattttagccttgcggagcagattaaatgagcacgtgtttcattaagattgatttaaatataattaatatttcaccactgtagttttaaaattgccttctaagtcatggttgatttttaataggtatcggctctaattagattactattttggtttttaaatgttatttctatttcatttgtaggctcgtttttaatgtgattttattagattctagttttaaaatcatcttttagccttaatgagcagactgaatgaacatgtttcattaagattaacttcattataatgaattatttaatcattctagtgggttataaatcagctctatttaaattaattgtcgtgtttatttctatttaatttttgattattttttcagggtcgtttttatgtgattttttcccgtttctaattttaaactaattgtttgatttttatatgattcgactctttttaaatgaattgttttgctttttttattgagcatatatatattatacaatttcaatcaacaattgttgtgctttttaaatgttgtttctatttcatttctagggtcgattttattcgattctagttttaaaatcgtgatttttaacttctaaccttactgagcagactgaatgaacatgtttcatcaagattaacttcattatagttaattattgaatcgtgctagtggtttataaatcagctctatttaaattaatcatcatcattatttctatttaatttttattatttctttaggattatttttatgtgatttttttcacgttctagttttaaactcaatgctcattactgctcacccttgctgagcagtctttcgttaagattaaattaattaataaccattctagttttaaaacagtgtcttaaggcatagctgatattttacaagtttactatttaatatatctcctttctttttttagggtcgatttcatctgatttgattaatttttattatcttcactaagctttttgttctaggtttatctcagtattattactgttatatttgaactgaatttctcctcatagtttattcattttatttatttattttttgtttattcgctattttgtattttagtttttaattagaaattttattagaatcgttttgaactagtttttatgcactctaacattaatattaaatcataactattatttttattataatttatttgtgtttatctttttgtgtagctttcatgtgtttttattttagttttattatgtttgcattgaattttttggactagtttatataacgatatacagaaaagccatgacaacaaatgtttttataatacctattaagtataagctaatcagatttccactattctgttcaaagttaacattattagccattctgatggatcatttgacatttgtcatatgtctttatgaacactgctctttcagatgccacttttaaagctgttttttacagtaaatgaacgtttattattatgacattatttcctaaggcctctagagtacagcaggatgtggcgtgtgcattactgtgtaattatgctgactgtaaatgtgcatttatagactgtcagcatgtctgactgtgcgctgttgtgatttgtcctgcacagaccaagggggatccactagttctagagcggccatcgaattgggatccaccggtcgccaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataaagcggcctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccctagagcatggctacgtaattcacgtcacgactccacccctccaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccagatccgctagagcggccgccaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 8 - DNA, 8364 bp, Artificial Sequence
  pZ?LFBG plasmid as a vector containing LFB
cagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacggtcgcccagcaattcctgcagcccgggggatctgtccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtcgacggtatcgataagcttgataattctgcgcgcgcagattccgtgtgggcctggtaatatcttaccattttaagctattttaagattagggaaaaaaattaaataaaactactttaaaattactttccttaaaaagcagcatatttatttagtttttgtttaactggctcaagattgtaatattttacttttacatgtttttttaagtacctaaatgtgttcaaaagtaaagtatgttattgtttttttttttttaatgttagctaagctttaaattgtaatatattacttttaaaagtgactttttaaaaatgtaaataaaaaagtaactataaatgtgtaatgcatgttatttctgtgggtttttaattttcatctcaactgggcttacaattttaacataataaaaatcaaatcaaatcaaatcaggcccttatttgtcacatacactcttgtatactgaaataaactaaatgttgagaaaaatttaaatacaccaaaataacgcataccttttttgtaattacctaagtaaaacatttagttccttttttaagtgactcaagattgtaatctattaattgtaaaagataaaactagtgatcaatctgacgtgcttctatgcagcctgtgagattaattgtgatgtttaattattaaacaatttaaatggatcaaacatttcacatgtgcttattgtgtccaacaaaaccagcagaagcacttaaaaatattgcatgacaacatgagtttttaatgtaaattatatatacttcctaaacgaagttaatcatatttctaaaataataatgtcattgaaatatttaaataaatgataagctttagttgattcaacataaaattgaaagcaataaggactttttttttttacagcggtgaagcaaaagcatgtaataatcatataatataataaataaataaataaataatggctcttggtgcaaatatgaacactttaaataaatatagaataaatatgatcaataagtcataacgacgtgttttatgttgattataatttaaacatgtttctaacgcaagttttaaaaactgttttaaagtattattttaagttataagatcaatttgactcaacattttgtagtgaaaatatgttgtgtaaaattaatataatatacactataaaaatgcagggttccacacaatacattaatgttgtcccaacacaaatcgattaagttaacaaatatatgtggattgaacataaaaaattaagttgtcccaatgaaatctcaagaattgtgttgtttcagctcattttaaataagtagttttaacaagtaaaaaataacatatttttgagtgtgtatatatatatattacaaaatataataatataaaatgtaaaagatataaaccatgctacatatatttaagagctattttgcacattacttttttgcaagccattttagtgttttttttacaaatatatattagctaaatatacaatatcttaaatatacaattaatcagacatgcaattaatgcatttaacctacataaaaatacatgatcaacatatgttttatgctgattacaactaaatcaaacgtaatgaaattaatttataaaagcgatttaaatatgtcaatattttaagttaaaatcacttgaaacctactataaatgaacttaaattaaaaaaatctcctttaattccaatgcaacagtgttgttttttcagctgaggacccccggtgcagatggttggcccccggcccgctattctaaggaagcctttcttaattttagggcggcgctgcatgcgctcagatcagattcacgggtggagagggttattttctcttttttattttttcttgcctgctcatatttgggcctgtgtgaaccccaggctccgcctcggcccccaggagccgctcttatttaaaggggatcctcgccttcagtcttcgtcccagtggaagcatcagtcttgtgataacaacttggtgagtcctacagaccagcgctcgactctaggctttaaatgcattgatttctctggattagctaccgggagagttttaactttgcttattagtcttgtgctcgaactgctaggtttaaagagtgttttctctaatgcatgcatgaagcgacagctgaaaaccagaaatagagcgtttgtcctcctgttaaactctagcctttctttggcatttgtcagtccgagtcttcagctgccggcaggagattgggtttcagtaacacttgctgagcacaaactacacgtgctttcttcagatttaattcattattaatcattctagatttaaatgatgttcttactcattgctgattattttagccttgcggagcagattaaatgagcacgtgtttcattaagattgatttaaatataattaatatttcaccactgtagttttaaaattgccttctaagtcatggttgatttttaataggtatcggctctaattagattactattttggtttttaaatgttatttctatttcatttgtaggctcgtttttaatgtgattttattagattctagttttaaaatcatcttttagccttaatgagcagactgaatgaacatgtttcattaagattaacttcattataatgaattatttaatcattctagtgggttataaatcagctctatttaaattaattgtcgtgtttatttctatttaatttttgattattttttcagggtcgtttttatgtgattttttcccgtttctaattttaaactaattgtttgatttttatatgattcgactctttttaaatgaattgttttgctttttttattgagcatatatatattatacaatttcaatcaacaattgttgtgctttttaaatgttgtttctatttcatttctagggtcgattttattcgattctagttttaaaatcgtgatttttaacttctaaccttactgagcagactgaatgaacatgtttcatcaagattaacttcattatagttaattattgaatcgtgctagtggtttataaatcagctctatttaaattaatcatcatcattatttctatttaatttttattatttctttaggattatttttatgtgatttttttcacgttctagttttaaactcaatgctcattactgctcacccttgctgagcagtctttcgttaagattaaattaattaataaccattctagttttaaaacagtgtcttaaggcatagctgatattttacaagtttactatttaatatatctcctttctttttttagggtcgatttcatctgatttgattaatttttattatcttcactaagctttttgttctaggtttatctcagtattattactgttatatttgaactgaatttctcctcatagtttattcattttatttatttattttttgtttattcgctattttgtattttagtttttaattagaaattttattagaatcgttttgaactagtttttatgcactctaacattaatattaaatcataactattatttttattataatttatttgtgtttatctttttgtgtagctttcatgtgtttttattttagttttattatgtttgcattgaattttttggactagtttatataacgatatacagaaaagccatgacaacaaatgtttttataatacctattaagtataagctaatcagatttccactattctgttcaaagttaacattattagccattctgatggatcatttgacatttgtcatatgtctttatgaacactgctctttcagatgccacttttaaagctgttttttacagtaaatgaacgtttattattatgacattatttcctaaggcctctagagtacagcaggatgtggcgtgtgcattactgtgtaattatgctgactgtaaatgtgcatttatagactgtcagcatgtctgactgtgcgctgttgtgatttgtcctgcacagaccaagggggatccactagttctagagcggccatcgaattgggatccaccgggatcccgccaccatgttcaaatgccgccgatggcagtggaggatgaagaagctgggtgctccctctatcacctgtgtgaggagggcctttatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataactcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccctagagcatggctacgtaattcacgtcacgactccacccctccaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccagatccgctagagcggccgccaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

(19) United States
(12) Patent Application Publication
(10) Pub. No.: US 2012/0054880 A1
(43) Pub. Date: Mar. 1, 2012

(54) METHOD FOR ENHANCING PRODUCTION OF PATHOGEN-RESISTANT PROTEINS USING BIOREACTOR

(75) Inventors: HUAI-JEN TSAI, TAIPEI CITY (TW); CHENG-YUNG LIN, TAIPEI CITY (TW)

(73) Assignee: NATIONAL TAIWAN UNIVERSITY, TAIPEI CITY (TW)

(21) Appl. No.: 13/216,396

(22) Filed: Aug. 24, 2011

(30) Foreign Application Priority Data
Aug. 24, 2010 (TW) 099128346

(51) Int. Cl. C12N 15/63

(52) U.S. Cl. 800/21; 435/320.1

(57) ABSTRACT

A method for enhancing production of pathogen-resistant proteins using bioreactor comprising: (a) constructing a plasmid which comprises a nucleic acid sequence of exogenous pathogen-resistant protein, a nucleic acid sequence of fluorescence protein, a nucleic acid sequence of protease-cutting site and an α-actin promoter; (b) transferring the constructed plasmid into fish embryo(s) of selected line after linearization; (c) domesticating the fish to enable to grow, mate and spawn; and (d) collecting fish egg(s), fish embryo(s) and adult fish(s) to obtain pathogen-resistant protein-rich organism(s) is described. A nucleic acid construct comprising an α-actin promoter, a nucleic acid sequence of exogenous pathogen-resistant protein and a nucleic acid sequence of fluorescence protein which are operably linked in the 5′ to 3′ direction is also presented. An expression vector comprising said nucleic acid construct is further described.

### FIELD OF THE INVENTION

The present invention relates to a method for enhancing production of pathogen-resistant proteins using bioreactor, a nucleic acid construct used therein and an expression vector comprising the same.

### BACKGROUND OF THE INVENTION

Lactoferrin, a member of the transferrin family, is an 80 kDa iron-binding glycoprotein. In mammals, lactoferrin is a major component of milk and is present in neutrophil granules and other exocrine secretions, such as tears, saliva, and cervical mucus. The functions of lactoferrin include the regulation of iron homeostasis, host defense against a broad range of microbial infections, anti-inflammatory activity, the regulation of cellular growth and differentiation, and even protection against cancer development and metastasis.

When bovine lactoferrin is hydrolyzed by pepsin in the digestive tract, the amphipathic bovine lactoferricin (LFB) is produced. This peptide is highly positively charged with a molecular mass of 3142-Da drived from the N terminus of bovine lactoferrin (from Phe17 to Phe41). LFB is an antimicrobial peptide that can kill or inactivate many pathogens, including Gramnegative bacteria, such as Escherichia coli, Klebsiella pneumoniae, Proteus vulgaris and Pseudomonas aeruginosa, as well as Gram-positive bacteria, such as Clostridium paraputrificum, Corynebacterium ammoniagenes, Enterococcus faecalis, Listeria monocytogenes and Streptococcus bovis. The antimicrobial activity of LFB originates from the arrangement of its amphipathic b-sheet where hydrophobic amino acid residues are located on one side and positively charged amino acid residues are located on the other side. For Gram-negative bacteria, LFB interrupts the integrity of the cell membrane by combining the positively charged LFB with the negatively charged lipopolysaccharides (LPS) in the outer membrane. For Gram-positive bacteria, whose cell membrane does not have LPS, it is thought that LFB combines with the teichoic acid layer surrounding the cytoplasmic membrane. As a consequence of LFB binding, the transmembrane electrochemical and pH gradients of bacteria are lost, which, in turn, results in cytolysis.

In addition to its antibacterial activity, LFB is also reported to have antimicrobial activity against such parasites as Eimeria stiedae, Giardia lamblia and Toxoplasma gondii, as well as against fungus, by disturbing the proton gradient across the cell membrane. LFB can also interact with heparin sulfate (HS) and glycosaminoglycans (GAGs), which results in blocking infection from viruses that bind to HS and GAGs on the cell membrane. The antiviral activity of LFB seems to be a consequence of the secondary structure formed by a highly positive charge and a disulfide bond. Because LFB can kill a wide variety of pathogens, it is an excellent antimicrobial peptide for application in the aquaculture environment.

For many reasons, zebrafish (Danio rerio) is an excellent model system. As a eukaryote, zebrafish can produce proteins with a complicated post-translational modification. Moreover, zebrafish are fecund, with each pair of parents producing 200e300 eggs each time. With a gestation period of three months, zebrafish can spawn eggs throughout the year under a temperature control set at 27˜29° C. and lay eggs once to twice a week. Two further merits convinced us to use zebrafish as a bioreactor to produce LFB. First, establishing a culture system for zebrafish is simple and inexpensive. Second, and most importantly, the manipulation of gene transfer for zebrafish is easy, allowing various transgenic lines to be screened and generated.

The CMV promoter has been used to drive human coagulation factor VII in zebrafish, African catfish, and tilapia fertilized eggs. A medaka β-actin promoter was constructed to drive goldfish luteinizing hormone in rainbow trout eggs. Recently, it was reported that using the CMV or mylz2 promoter to drive epinecidin-1 in adult zebrafish muscle through injection and electroporation. These reports only focused on the transient expression of the transferred genes, either in fish embryos or in fish muscle, resulting in the mosaic-like and temporary expression of foreign genes. However, in order to serve as a bioreactor, it is necessary to generate transgenic lines that can produce heterologous proteins so that 1) the expression level remains constant, 2) the amount of recombinant protein is high and predictable, and 3) the large-scale culture of progeny from a close genetic background is accessible.

The present inventor published a paper regarding muscle and egg of zebrafish as bioreactors in Nov. 26, 2009. In said paper, the LFB protein generating technology using β actin promoter to drive gene expression of exogenous pathogen-resistant protein was disclosed (Cheng-Yung Lin, Ping-His Yang, Chia-Lin Kao, Han-1Huang and Huai-Jen Tsai. Transgenic zebrafish eggs containing bactericidal peptide is a novel food supplement enhancing resistance to pathogenic infection of fish. Fish & Shellfish Immunology 2010; 28:419-427). Through this technology, the exogenous pathogen-resistant protein can be expressed in all cells including eggs. The gene of pathogen-resistant protein also can be inherited by the progeny rather than only transiently expressed in F0 individuals. The pathogen-resistant protein produced by transgenic fish can be readily utilized as feed or additive without further purification of the recombinant protein. However, this disclosed technology is still defective in yield. More importantly, the exogenous protein generated by every cell should impact on the physiology of fish. Therefore, there is a need to efficiently supply power to higher production by a specific tissue to promote the utilization efficiency and healthy.

### BRIEF DESCRIPTION OF THE DRAWINGS

The invention can be more fully understood by reading the subsequent detailed descriptions and examples with references made to the accompanying drawing, wherein:

FIG. 1 shows the scheme of plasmid construction according to one embodiment of the present invention. The completed plasmid comprises Kozak sequence, α-actin promoter, exogenous pathogen-resistant protein sequence, fluorescence protein sequence and cutting site of various enzymes.

FIG. 2 shows the nucleic acid construct according to one embodiment of the present invention. The nucleic acid construct comprises Kozak sequence, α-actin promoter, exogenous pathogen-resistant protein sequence and fluorescence protein sequence. It can further comprise post-translational enzyme cutting sites (for example, pepsin cutting site).

FIG. 3 shows the fluorescence distribution of GFP expressed through the whole embryo at 72 hpf according to one embodiment of the present invention. (A) The fluorescence was ubiquitously expressed through all muscle cells of the whole exbryo; for example in (B) head muscle, (C) pectoral fin and muscle of the body wall, and (D) muscle of the truncus.

FIG. 4 shows comparison of fluorescence level between condition drived by α-actin 1a promoter and condition drived by β-actin 1 promoter under various exposures according to one embodiment of the present invention.

### SUMMARY OF THE INVENTION

The present invention relates to a method for enhancing production of pathogen-resistant proteins using bioreactor comprising: (a) constructing a plasmid which comprises a nucleic acid sequence of exogenous pathogen-resistant protein, a nucleic acid sequence of fluorescence protein, a nucleic acid sequence of protease-cutting site and an α-actin promoter; (b) transferring the constructed plasmid into fish embryo(s) of selected line after linearization; (c) domesticating the fish to enable to grow, mate and spawn; and (d) collecting fish egg(s), fish embryo(s) and adult fish(s) to obtain pathogen-resistant protein-rich organism(s). The present invention also relates to a nucleic acid construct comprising an α-actin promoter, a nucleic acid sequence of exogenous pathogen-resistant protein and a nucleic acid sequence of fluorescence protein which are operably linked in the 5′ to 3′ direction. The present invention further relates to an expression vector comprising a nucleic acid construct which comprises an α-actin promoter, a nucleic acid sequence of exogenous pathogen-resistant protein and a nucleic acid sequence of fluorescence protein which are operably linked in the 5′ to 3′ direction.

### DETAILED DESCRIPTION OF THE INVENTION

As used hereinafter, the term “pathogen-resistant protein(s)” refers to proteins or peptides with specific secondary structures. These proteins or peptides can be bactericide, fungicidal and/or virucidal. There are various pathogen-resistant proteins and each of them may works through different mechanisms. For example, a pathogen-resistant protein can cause death of a Gram (−) bacterium by binding to the bacterial cell wall and/or cell membrane and making pores thereon.

As used hereinafter, the term “bioreactors” refers to organisms used for producing exogenous useful proteins, which includes bacteria, cell lines, animals and plants.

As used hereinafter, the term “exogenous” refers to any DNA, RNA, protein or substance that originates outside of the organism of concern or study.

As used hereinafter, the term “transferring” and/or “transfer” refer to delivering the exogenous material, such as DNA, RNA or protein, into an organism by microinjection, electroporation or any other method that can be recognized by one skilled in the art.

As used hereinafter, the term “skeletal muscle” refers to a form of striated muscle tissue existing under control of the somatic nervous system. That is, it is voluntarily controlled. Most skeletal muscles are attached to bones by bundles of collagen fibers known as tendons.

As used herein, the term “a” or “an” are employed to describe elements and components of the invention. This is done merely for convenience and to give a general sense of the invention. This description should be read to include one or at least one and the singular also includes the plural unless it is obvious that it is meant otherwise.

As used herein, the term “or” is employed to describe “and/or”.

Accordingly, the present invention provides a method for enhancing production of pathogen-resistant proteins using bioreactor comprising: (a) constructing a plasmid which comprises a nucleic acid sequence of exogenous pathogen-resistant protein, a nucleic acid sequence of fluorescence protein, a nucleic acid sequence of protease-cutting site and an α-actin promoter; (b) linearizing the constructed plasmid; (c) transferring the linearized constructed plasmid into fish embryo(s); (d) culturing the fish embryo(s) to enable to grow, mate and spawn; and (e) collecting fish egg(s), fish embryo(s) and adult fish(s) drived from step (d) to obtain pathogen-resistant protein-rich organism(s).

In one embodiment of the present invention, the fish egg(s), fish embryo(s) and adult fish(es) is the egg(s), embryo(s) and adult fish(es) of zebrafish. In another embodiment of the present invention, the fish egg(s), fish embryo(s) and adult fish(es) is the egg(s), embryo(s) and adult fish(es) of freshwater fishes including catfish, tilapia, goldfish, or carp, medaka, cichlidae and rainbow trout. In still another embodiment of the present invention, the fish egg(s), fish embryo(s) and adult fish(es) is the egg(s), embryo(s) and adult fish(es) of saltwater fishes including salmon, grouper, milkfish, flounder, seabass and seabream. In another embodiment of the present invention, the fish egg(s), fish embryo(s) and adult fish(es) is the egg(s), embryo(s) and adult fish(es) of ornamental fishes.

In one embodiment of the present invention, the nucleic acid sequence of exogenous pathogen-resistant protein is lactoferrin nucleic acid sequence. In another embodiment of the present invention, the protease-cutting site is pepsin-cutting site. In still another embodiment of the present invention, the nucleic acid sequence of fluorescence protein is nucleic acid sequence of fluorescence protein with green, red, yellow, blue, indigo or other colors.

In one embodiment of the present invention, the exogenous pathogen-resistant protein is specific expressed in skeletal muscle.

The present invention also provides a nucleic acid construct comprising an α-actin promoter, a nucleic acid sequence of exogenous pathogen-resistant protein and a nucleic acid sequence of fluorescence protein which are operably linked in the 5′ to 3′ direction.

In one embodiment of the present invention, the nucleic acid sequence of exogenous pathogen-resistant protein is lactoferrin nucleic acid sequence. In another embodiment of the present invention, the nucleic acid sequence of fluorescence protein is nucleic acid sequence of fluorescence protein with green, red, yellow, blue, indigo or other colors.

In one embodiment of the present invention, the nucleic acid construct further comprises a nucleic acid sequence of protease-cutting site. In another embodiment of the present invention, the protease-cutting site is pepsin-cutting site.

The present invention further provides an expression vector comprising a nucleic acid construct which comprises an α-actin promoter, a nucleic acid sequence of exogenous pathogen-resistant protein and a nucleic acid sequence of fluorescence protein which are operably linked in the 5′ to 3′ direction.

In one embodiment of the present invention, the nucleic acid sequence of exogenous pathogen-resistant protein is lactoferrin nucleic acid sequence. In another embodiment of the present invention, the nucleic acid sequence of fluorescence protein is nucleic acid sequence of fluorescence protein with green, red, yellow, blue, indigo or other colors.

In one embodiment of the present invention, the expression vector further comprises a nucleic acid sequence of protease-cutting site. In another embodiment of the present invention, the protease-cutting site is pepsin-cutting site.

According to certain embodiments, the present invention a method for enhancing production of pathogen-resistant proteins using either adults or embryos drived from a stable transgenic line of fish as a bioreactor. The expression level of exogenous pathogen-resistant proteins produced by each embryo or adult from the fish transgenic line is high and quite close among all siblings Importantly, the foreign protein is produced during the entire lifetime of the transgenic fish. The adult transgenic fish can thus accumulate a very high level of exogenous pathogen-resistant proteins. As a consequence, these transgenic fish can be directly mixed with fish pellet without further purification of the target protein. Alternatively, a large number of embryos spawned from a transgenic line can be directly applied in fish pellets without sacrificing the parental generation. Thus, either adult fish or embryos can be used as a feed addictive.

The next examples provide some exemplary embodiments of the present invention as follows:

### EXAMPLES

The examples below are non-limiting and are merely representative of various aspects and features of the present invention.

### Example 1

### Nucleic Acid Construct and Construction of Expression Vector

The LFB cDNA that contains kozak sequence fused with GFP (green fluorescent protein) cDNA (LFB-GFP) was obtained by a sequential PCR amplification from the GFP cDNA template (SEQ ID NO:1), starting with the first set of primer pairs, LFBTG-F1 (SEQ ID NO: 4) and LFBTG-R (SEQ ID NO: 3), the second set of primer pairs, LFBTG-F2 (SEQ ID NO: 5) and LFBTG-R, and then the third set of primer pairs, LFBTG-F3 (SEQ ID NO: 6) and LFBTG-R. The final PCR product should be LFB-GFP which is expected to be 789 bp.

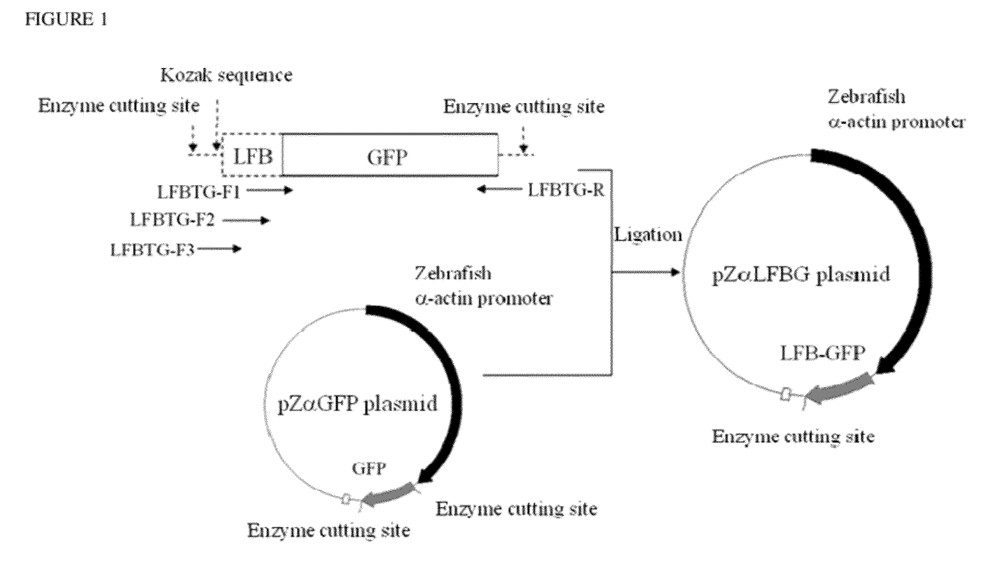

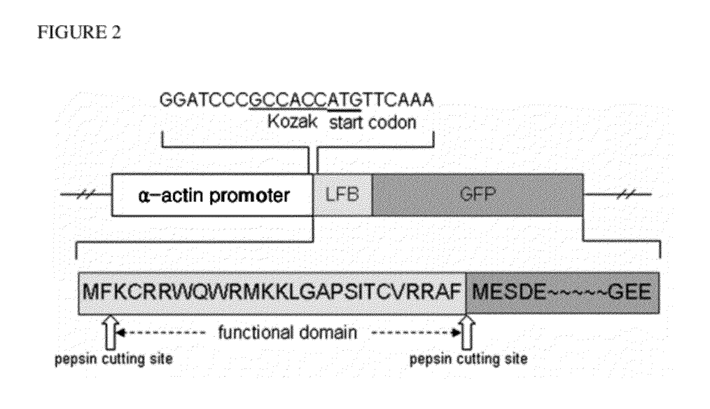

The LFB-GFP was digested with BamHI/XhoI and ligated with the BamHI/XhoI-cut pZαGFP (SEQ ID NO: 7) to generate pZαLFBG (SEQ ID NO: 8) (FIG. 1). Location of features within the pZαLFBG is as follows: ITR-R at 260-396 bp; zebrafish α-actin promoter at 473-4478 bp; start codon of LFB-TGFP gene at 4491-4493 bp; stop codon of LFB-TGFP gene at 5265-5267 bp; fragment containing SV40 poly-A signal sequence at 5358-5410 bp; and ITR-L at 5555-5691 bp. The backbone vector of pZαLFBG is pBluescript. The nucleic acid construct comprised by the expression vector is shown in FIG. 2. The α-actin 1a promoter was used in the pZαLFBG plasmid, the pathogen-resistant gene therefore can be stably expressed in the muscle cells.

### Example 2

### Gene Transformation of the Zebrafish

### A. Breeding of Zebrafish:

The wild-type zebrafish (Danio rerio) were grew for two month and cultured in thermostatic incubator under 28.5° C. with 14 hr/10 hr light/dark cycle. The ratio of male and female adult zebrafish was 2:3 with a total of 40 fishes. The zebrafishes were breeded in 60 cm×20 cm×30 cm glass aquarium and feeded with artificial dry fish feed and brine shrimp.

### B. Collection of Fertilized Eggs:

At 11 PM of the day before microinjection, the zebrafishes were collected in pet box and isolated with isolation net before entering the dark cycle. The fish eggs were collected every 15 to 20 min from the beginning of light cycle in the next day. 30 to 40 fish eggs could be collected each time, and total 250 to 300 eggs could be collected in one experiment.

### C. Microinjection:

After linearization of the plasmid to be injected, the double distilled water was added to adjust the concentration thereof. The stock solution of 5× phenol red in phosphate buffered saline (0.005 gram per ml) was then mixed into said plasmid solution and diluted to the desired injection concentration of 25 ng/ml plasmid DNA in 1% phenol red. The plasmid solution was sucked into the capillary with a diameter of 10 to 15 μm by using of zebrafish microinjector (Drummond). Approximately 2.3 nl of solution was injected into a one-cell stage of zebrafish embryo each time. After gene transfer, the fertilized eggs were rinsed with sterile water and incubated in thermostatic incubator under 28.5° C. The eggs and embryos were cultured and observed to adulthood as the putative transgenic founders F0.

### Example 3

### Transgenic zebrafish analysis, transgenic line establishing and yield or product standardization

A. Analysis and screening:

### (a) Genomic DNA Extraction:

100 two day-old embryos were added with 500 μl lysis buffer (including 10 mM pH8.0 TrisCl, 10 mM EDTA, 200 mM NaCl and 0.5% SDS) and 200 μg/ml Proteinase K. The mixture was put under 55° C. to react for 2 hr. After extracting DNA with phenol/chloroform mixture, 2× volumn of anhydrous alcohol was added. In the following, the mixture was subjected to 14,000 rpm centrifugation under 4° C. for 5 min. The resulting DNA precipitation was then washed with 1 ml 70% ethanol followed by 14,000 rpm centrifugation under 4° C. for 5 min. The final precipitation was air dried, dissolved in 500 μl TE buffer and stored in −20° C.

### (b) PCR Analysis:

1 μl 10 mM dNTPs, 2.5 μl 10×PCR buffer (including 1.5 mM MgCl₂), 1 μl 5 μM forward primer, 1 μl 5 μM reverse primer, 1 μl DNA template (1 ng cyclic plasmid as the control) were sequentially added into a 0.5 ml eppendorf. Steril water was then added to the solution to a volumn of 25 μl. After mixing and adding with 1 drop of mineral oil, the eppendorf was put into the PCR machine (DNA Thermal Cycle model 480, Perkin Elmer). The reaction condition was as follows: reacting under 94° C. for 10 min, adding 1 μl commercial proteinase Prozyme (2 unit/μl), dissociating DNA under 94° C. for 1 min, annealing DNA under 60° C. for 1 min and elongating DNA under 72° C. for 2 min (cycle). The reaction totally contained 30 cycles and completed with final reaction under 72° C. for 10 min. 10 μl reaction product was analyzed by 0.8% agarose gel electrophoresis.

### B. Inheritance:

The microinjected embryos were placed in concavity slide containing a small amount of water. The heart development was observed by using of microscope (Leica MZ12). The survival larvaes were moved to aquarium and incubated therein. These larvaes were fed with artificial rotifer (OSI) few times a day. About 1 week later, two feeds a day were given on Brine shrimp. About 10 weeks later, sex maturation can be reached. The parent fishes were mated with wild-type fishes in a ratio of 1:1. Those parent fishes, which were capable of generate F1 progeny with the transgene, were kept; the F1 progeny was bred for following analysis. The stable transgenic line was named as ZαL-3.

### C. Yield or Product Standardization

The zebrafish line bringing the exogenous transgene was subjected to mass production in large aquarium. After proliferation of the transgenic line, the heterozygotic line was mated to generate the homozygotic line. Therefore, each progeny of the following generation from the homozygotic line will carry the transgene. In addition, the transgenic line needs to be intercross with the wild-type strain in a due course to maintain their health.

### Example 4

### Increasing of Exogenous Pathogen-Resistant Protein Expression by α-Actin Promoter

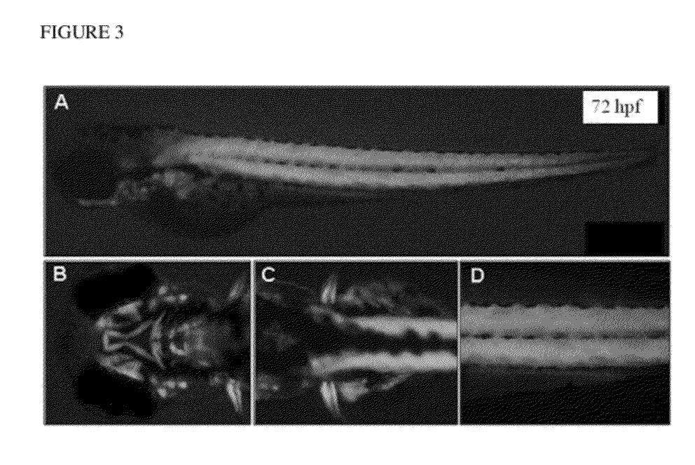

To obtain the stable transgenic line ZαL3, the zebrafish embryos were injected with linearized recombinant plasmid pZαLFBG comprising α-actin 1a promoter, gene of exogenous pathogen-resistant protein LFB and gene of green fluorescent protein GFP. The GFP expression of 72-hpf embryos was observed under microscope. The results were shown in FIG. 3. The GFP tabled all the skeletal muscles through the whole body (FIG. 3A), such as the head muscle (FIG. 3B), pectoral fin and body wall muscle (FIG. 3C) and truncus muscle (FIG. 3D). The expression level of the GFP was proportional to the intensity of the green fluorescence, which also indicated the expression level of the pathogen-resistant protein. It was shown that the exogenous pathogen-resistant protein was specifically and highly expressed in all the skeletal muscles through the whole transgenic zebrafish body.

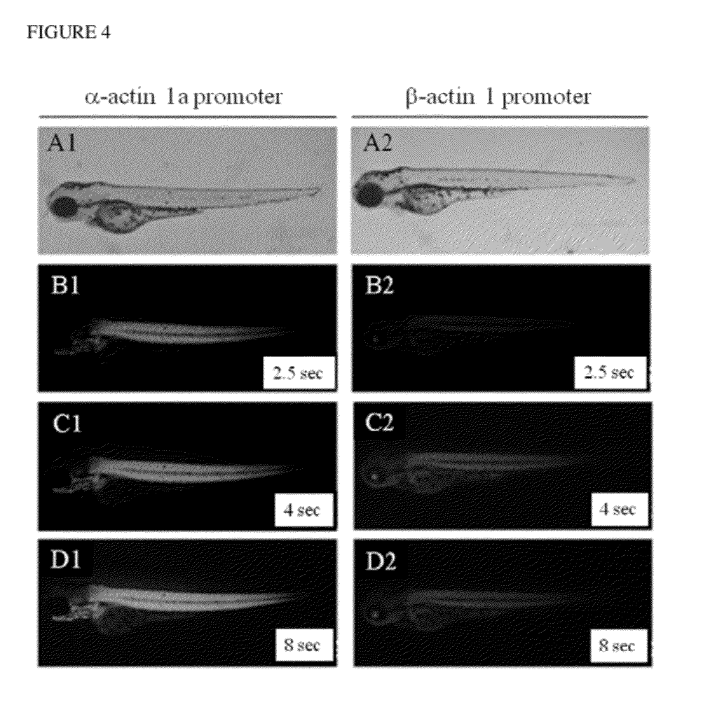

The expression level of GFP drived by α-actin 1a promoter and β-actin promoter were compared in 72-hpf stable transgenic lines of zebrafish (FIG. 4 A1 and A2) under the same exposure condition. The results of comparison were shown in FIG. 4. When under exposure of 2.5 sec (FIG. 4 B1 and B2), 4 sec (FIG. 4 C1 and C2) or 8 sec (FIG. 4 D1 and D2), the intensities of the green fluorescence drived by α-actin 1a promoter were stronger than that of drived by β-actin promoter. These suggested that replacing β-actin promoter with α-actin 1a promoter improved efficiency of the zebrafish bioreactors and significantly increased yield of the exogenous pathogen-resistant protein.

One skilled in the art readily appreciates that the present invention is well adapted to carry out the objects and obtain the ends and advantages mentioned, as well as those inherent therein. The animals, processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A method for enhancing production of pathogen-resistant proteins using bioreactors comprising:
(a) constructing a plasmid which comprises a nucleic acid sequence of exogenous pathogen-resistant protein, a nucleic acid sequence of fluorescence protein, a nucleic acid sequence of protease-cutting site and an α-actin promoter;
(b) linearizing the constructed plasmid;
(c) transferring the linearized plasmid into fish embryo(s);
(d) culturing the fish embryo(s) to enable to grow, mate and spawn; and
(e) collecting fish egg(s), fish embryo(s) and adult fish(es) drived from step (d) to obtain pathogen-resistant protein-rich organism(s).

2. The method of claim 1, wherein the nucleic acid sequence of exogenous pathogen-resistant protein is lactoferrin nucleic acid sequence.

3. The method of claim 1, wherein the protease-cutting site is pepsin-cutting site.

4. The method of claim 1, wherein the nucleic acid sequence of fluorescence protein is nucleic acid sequence of fluorescence protein with green, red, yellow, blue, indigo or other colors.

5. The method of claim 1, wherein the exogenous pathogen-resistant protein is specific expressed in skeletal muscle.

6. A nucleic acid construct comprising an α-actin promoter, a nucleic acid sequence of exogenous pathogen-resistant protein and a nucleic acid sequence of fluorescence protein which are operably linked in the 5′ to 3′ direction.

7. The nucleic acid construct of claim 6, wherein the nucleic acid sequence of exogenous pathogen-resistant protein is lactoferrin nucleic acid sequence.

8. The nucleic acid construct of claim 6, wherein the nucleic acid sequence of fluorescence protein is nucleic acid sequence of fluorescence protein with green, red, yellow, blue, indigo or other colors.

9. The nucleic acid construct of claim 6, which further comprises a nucleic acid sequence of protease-cutting site.

10. The nucleic acid construct of claim 9, wherein the protease-cutting site is pepsin-cutting site.

11. An expression vector comprising a nucleic acid construct which comprises an α-actin promoter, a nucleic acid sequence of exogenous pathogen-resistant protein and a nucleic acid sequence of fluorescence protein which are operably linked in the 5′ to 3′ direction.

12. The expression vector of claim 11, wherein the nucleic acid sequence of exogenous pathogen-resistant protein is lactoferrin nucleic acid sequence.

13. The expression vector of claim 11, wherein the nucleic acid sequence of fluorescence protein is nucleic acid sequence of fluorescence protein with green, red, yellow, blue, indigo or other colors.

14. The expression vector of claim 11, which further comprises a nucleic acid sequence of protease-cutting site.

15. The expression vector of claim 14, wherein the protease-cutting site is pepsin-cutting site.
